(19) United States
(12) Patent Application Publication
(10) Pub. No.: US 2021/0009624 A1
(43) Pub. Date: Jan. 14, 2021

(54) [18F] FMAU LABELING FOR PET IMAGING OF CANCER PATIENTS

(71) Applicant: University of Southern California, Los Angeles, CA (US)

(72) Inventors: Kai Chen, Los Angeles, CA (US); Peter S. Conti, Los Angeles, CA (US)

(21) Appl. No.: 16/980,774

(22) PCT Filed: Mar. 29, 2019
(86) PCT No.: PCT/US2019/024928

§ 371 Date: Sep. 14, 2020

Related U.S. Application Data
(60) Provisional application No. 62/650,939, filed on Mar. 30, 2018.

(51) Int. Cl. C07H 19/06; B01J 31/02; C07B 59/00; A61K 51/04

(52) U.S. Cl. CPC C07H 19/06; B01J 31/0275; C07B 2200/05; A61K 51/0491; C07B 59/005

(57) ABSTRACT

Provided herein are methods and labeling kits for synthesizing 2′-deoxy-2′-[¹⁸F]fluoro-5-methyl-1-beta-D-arabino-furanosyl-uracil in a one-pot reaction in compliance with CGMP. Also disclosed are labeling kits that can be assembled in an automated synthesis system to enable such a reaction.

### CROSS-REFERENCE TO RELATED APPLICATIONS

This application claims the benefit of priority under 35 U.S.C. § 119(e) of U.S. Application Ser. No. 62/650,939, filed Mar. 30, 2018. The disclosure of the prior application is considered part of and is incorporated by reference in the disclosure of this application in its entirety.

### FIELD OF THE INVENTION

The present invention relates generally to the synthesis of 2′-deoxy-2′[¹⁸F]fluoro-5-methyl-1-beta-D-arabinofuranosyl-uracil ([¹⁸F]FMAU), and more specifically to labeling kits for synthesis of [¹⁸F]FMAU in automated synthesis systems.

### BACKGROUND INFORMATION

Increased cellular proliferation is an integral part of the cancer phenotype. Rate of cellular proliferation or tumor growth is often measured via in vitro assays, which require biopsies that are difficult to obtain over time and in different areas of the body in patients with multiple metastatic lesions.

Some of these problems were eased through efforts for developing imaging methods to noninvasively measure the rate of tumor cell proliferation, for example by using Positron Emission Tomography (PET) in conjunction with tracers (e.g., tracers for the thymidine salvage pathway of DNA synthesis).

Potential imaging agents for these and a variety of other applications include antiviral and antileukemic nucleoside derivatives that are obtained through radiosynthesis. Such agents include [¹²⁵I] 2′-fluoro-5-iodo-1-beta-D-arabinofuranosyl-cytosine (FIAC), [¹²⁵I, ¹³¹I, ¹²³I] 2′-fluoro-5-iodo-1-beta-D-arabinofuranosyl-uracil (FIAU), 2′-deoxy-1-[¹¹C]methyl-pseudouridine, [methyl-¹¹C]3′-azido-thymidine, and 2′-fluoro-5-[methyl-¹¹C]-1-beta-D-arabinofuranosyl-uracil [¹¹C]FMAU.

Among the agents, [¹¹C]FMAU appears to be one of the best choices for a non- or minimally catabolized in vivo radiotracer of cellular proliferation. However, the procedure to prepare [¹¹C]FMAU involves formation of a dilithio compound, which makes the production complicated, hard to control, and unreliable. In addition, the short half-life of ¹¹C limits its clinical application. In contrast, ¹⁸F has a half-life of 120 min and the synthetic procedure for [¹⁸F]FMAU can be better controlled.

Thus, there is a need for improved methods for preparing imaging agents.

This background information is provided for the purpose of making known information believed by the applicant to be of possible relevance to the present invention. No admission is necessarily intended, nor should be construed, that any of the preceding information constitutes prior art against the present invention.

### SUMMARY OF THE INVENTION

The present invention is based, in part, on the development of a method and labeling kit for synthesis of [¹⁸F]FMAU. In particular, the labeling kit is assembled in an automated synthesis system, which allows tuning reactions conditions at each step of the synthesis. Some of the reaction factors are solvent effects, concentration effects, reaction time, and reaction temperature. The labeling kit enables automated [¹⁸F]FMAU synthesis in full compliance with cGMP and thus facilitates [¹⁸F]FMAU PET imaging in cancer patients. This is in contrast to the previously available semi-automated systems that could not be in compliance with cGMP requirements.

In one embodiment, the invention provides a system for producing 2′-deoxy-2′-[¹⁸F]fluoro-5-methyl-1-beta-D-arabinofuranosyl-uracil ([¹⁸F]FMAU) including 2-trifluoromethane-sulfonyl-1,3,5-tri-O-benzoyl ribofuranose; 2,4-bis-trimethylsilyl-5-methyl-uracil, a Friedel-Crafts catalyst, and hexamethyldisilizane; an eluent; an inlet for receiving [¹⁸F]-fluoride produced via a cyclotron; and an [¹⁸F]FMAU collection device.

In one aspect, the Friedel-Crafts catalyst is trimethylsilyl trifluoromethanesulfonate (TMSOTf). In various aspects, the system may be configured for automated one-pot synthesis. In many aspects, the system is in compliance with Current Good Manufacturing Practices (CGPMs). In some aspects, the system further includes tetrabutylammonium fluoride and acetonitrile. In other aspects, the system further includes sodium methoxide and methanol. In addition, in some aspects, the system further includes a carrier, excipient, diluent, or a combination thereof.

In another embodiment, the invention provides an automated synthesis module (ASM) for synthesizing [¹⁸F]FMAU including a first container for holding 2-trifluoromethane-sulfonyl-1,3,5-tri-O-benzoyl ribofuranose; a second container for holding 2,4-bis-trimethylsilyl-5-methyl-uracil, a Friedel-Crafts catalyst, and hexamethyldisilizane; a third container for holding an eluent; an inlet for receiving [18F]-fluoride produced via a cyclotron; and a fourth container for collecting [18F]FMAU.

In some aspects, the ASM further includes a fifth container for holding tetrabutylammonium fluoride and acetonitrile. In other aspects, the ASM further includes a sixth container for holding sodium methoxide and methanol. In some aspects, the ASM further includes a seventh container for holding carrier, excipient, diluent, or a combination thereof.

In an additional embodiment, the invention provides a method of synthesizing [¹⁸F]FMAU in a one-pot reaction including incubating 2-trifluoromethane-sulfonyl-1,3,5-tri-O-benzoyl ribofuranose with an [¹⁸F]-containing compound, thereby generating 2-[¹⁸F]fluoro-1,3,5-tri-O-benzoyl ribofuranose; incubating the 2-[¹⁸F]fluoro-1,3,5-tri-O-benzoyl ribofuranose with a solution containing 2,4-bis-trimethylsilyl-5-methyl-uracil, a Friedel-Crafts catalyst, and hexamethyldisilizane, thereby generating a mixture; and purifying the mixture via HPLC, thereby obtaining [¹⁸F]FMAU.

In various aspects, the method further includes, before purifying the mixture via HPLC, incubating the mixture with sodium methoxide and methanol to remove benzoyl groups. In other aspects, the method further includes adding a carrier, excipient, diluent, or a combination thereof to the [¹⁸F]FMAU. In some aspects, the method further includes diluting a solution of the [¹⁸F]FMAU to less than or equal to about 25 mCi per unit dose. In one aspect, the method is performed in a CGMP-compliant environment. In other aspects, the method is performed in an automated synthesis module. In many aspects, the [¹⁸F]-containing compound is [¹⁸F]tetrabutylammonium fluoride.

In yet another embodiment, the invention provides a method of screening conditions for GMP-compliant one-pot synthesis of [¹⁸F]FMAU including incubating in multiple ASMs or in one ASM at different times, an amount of 2-trifluoromethane-sulfonyl-1,3,5-tri-O-benzoyl ribofuranose with an amount of [18F]-containing compound, thereby generating an amount of 2-[18F]fluoro-1,3,5-tri-O-benzoyl ribofuranose; incubating, in each of the multiple ASMs or in one ASM at each of the different times, an amount of the 2-[18F]fluoro-1,3,5-tri-O-benzoyl ribofuranose with a solution containing an amount of 2,4-bis-trimethylsilyl-5-methyl-uracil, an amount of a Friedel-Crafts catalyst, and an amount of hexamethyldisilizane using pre-selected solvents, solute concentrations, incubation times, or temperatures thereby generating an amount of a mixture; purifying, in each of the multiple ASMs or in one ASM at each of the different times, an amount of the mixture via HPLC, thereby obtaining an amount of [18F]FMAU; and determining the amount of [¹⁸F]FMAU obtained using each of the pre-selected solvents, solute concentrations, incubation times, or temperatures.

In some aspects, the screening methods are performed under one-pot synthesis conditions. In some aspects, the screening methods further include, before purifying the mixture via HPLC, incubating the mixture with sodium methoxide and methanol to remove benzoyl groups.

In one embodiment, the invention provides a method of constructing a labeling system for obtaining [¹⁸F]FMAU including providing a first container of the system containing 2-trifluoromethane-sulfonyl-1,3,5-tri-O-benzoyl ribofuranose; providing a second container of the system containing 2,4-bis-trimethylsilyl-5-methyl-uracil, a Friedel-Crafts catalyst, and hexamethyldisilizane, wherein the second container is disposed to communicate with the first container; providing a third container of the system containing an eluent, wherein the third container is disposed to communicate with the first container; connecting to the system an inlet for receiving [18F]-fluoride produced via a cyclotron, wherein the inlet is disposed to communicate with the third container; and providing a fourth container of the system for collecting [18F]FMAU, wherein the fourth container is disposed to communicate with the second container.

In one aspect, the method further includes providing a fifth container of the system containing tetrabutylammonium fluoride and acetonitrile, wherein the fifth container is disposed to communicate with the first container. In another aspect, the method further includes providing a sixth container of the system containing sodium methoxide and methanol, wherein the sixth container is disposed to communicate with the third container. In some aspects, the method further includes providing a seventh container of the system containing a carrier, excipient, diluent, or a combination thereof, wherein the seventh container is disposed to communicate with the fourth container.

In another embodiment, the invention provides a method of detecting cellular proliferation via PET imaging including incubating 2-trifluoromethane-sulfonyl-1,3,5-tri-O-benzoyl ribofuranose with an [18F]-containing compound, thereby generating 2-[18F]fluoro-1,3,5-tri-O-benzoyl ribofuranose; incubating the 2-[¹⁸F]fluoro-1,3,5-tri-O-benzoyl ribofuranose with a solution containing 2,4-bis-trimethylsilyl-5-methyl-uracil, a Friedel-Crafts catalyst, and hexamethyldisilizane, thereby generating [¹⁸F]FMAU; administering the [¹⁸F]FMAU to a subject; and detecting the [¹⁸F]FMAU by imaging an area of the subject via PET. In some aspects, the [¹⁸F]FMAU is administered to the subject at less than or equal to 25 mCi per unit dose.

The embodiments described above have various advantages. For example, the production of [¹⁸F]FMAU can be accomplished in an easy to control and reliable manner and the half-life of ¹⁸F improves the clinical use of FMAU, for example for quantifying cell proliferation in cancer patients, as compared to [¹¹C]FMAU. In addition, the use of an automated synthesis system enables investigation of multiple parameters, such as solvent effects, concentration effects, reaction times, and reaction temperatures, so as to enable optimization of the overall reaction. The labeling kit also allows employment of a CGMP-compliant environment for the synthesis of [¹⁸F]FMAU.

Other aspects and advantages of the invention will be apparent from the following description and the appended claims.

### BRIEF DESCRIPTION OF THE DRAWINGS

The foregoing and other objects of the present disclosure, the various features thereof, as well as the disclosure itself may be more fully understood from the following description, when read together with the accompanying drawings in which:

FIG. 1 is a schematic representation of an embodiment of a process for one-pot synthesis of [¹⁸F]FMAU.

FIG. 2 is a set of microPET/CT images of a mouse bearing MDA-MB-231 breast tumor at 1 hr. post-injection of [¹⁸F]FMAU.

FIG. 3A is a set of PET images of [¹⁸F]FMAU in breast cancer patients in a first case.

FIG. 3B is a set of PET images of [¹⁸F]FMAU in breast cancer patients in a second case.

FIG. 4 is a schematic representation of an embodiment of the labeling kit for the automated manufacture of [¹⁸F]FMAU in an environment that fully complies with cGMP.

### DETAILED DESCRIPTION OF THE INVENTION

The present invention is based in part on the discovery that [¹⁸F]FMAU is one of the best radiotracers for detecting cellular proliferation, and that it is possible to carry out its radiosynthesis in a one-pot reaction.

The disclosures of any publications, patents, and patent applications referred to herein are hereby incorporated by reference in their entireties into this application to the same extent as if each individual publication, patent, or patent application was specifically and individually indicated to be incorporated by reference. The instant disclosure will govern in the instance that there is any inconsistency between the publications, patents, or patent applications and this disclosure.

Unless defined otherwise, all technical and scientific terms used herein have the same meaning as commonly understood by one of ordinary skill in the art to which this disclosure belongs. The initial definition provided for a group or term herein applies to that group or term throughout the present specification individually or as part of another group, unless otherwise indicated.

Before the present compositions and methods are described, it is to be understood that this invention is not limited to particular compositions, methods, and experimental conditions described, as such compositions, methods, and conditions may vary. It is also to be understood that the terminology used herein is for purposes of describing particular embodiments only, and is not intended to be limiting, since the scope of the present invention will be limited only in the appended claims.

Unless defined otherwise, all technical and scientific terms used herein have the same meaning as commonly understood by one of ordinary skill in the art to which this invention belongs. Although any methods and materials similar or equivalent to those described herein can be used in the practice or testing of the invention, it will be understood that modifications and variations are encompassed within the spirit and scope of the instant disclosure. The preferred methods and materials are now described.

As used in this specification and the appended claims, the singular forms “a”, “an”, and “the” include plural references unless the context clearly dictates otherwise. Thus, for example, references to “the method” includes one or more methods or steps of the type described herein, which will become apparent to persons skilled in the art upon reading this disclosure.

The term “about” or “approximately” are defined as being close to as understood by one of ordinary skill in the art, and in one non-limiting embodiment the terms are defined to be within 10%, preferably within 5%, more preferably within 1%, and most preferably within 0.5% of the qualified value.

The term “substantially” and its variations are defined as being largely but not necessarily wholly what is specified as understood by one of ordinary skill in the art, and in one non-limiting embodiment substantially refers to ranges within 10%, within 5%, within 1%, or within 0.5% of the qualified value.

The term “effective” as that term is used in the specification and/or claims, means adequate to accomplish a desired, expected, or intended result.

In one embodiment, the invention provides a system for producing 2′-deoxy-2′-[¹⁸F]fluoro-5-methyl-1-beta-D-arabinofuranosyl-uracil ([¹⁸F]FMAU) including 2-trifluoromethane-sulfonyl-1,3,5-tri-O-benzoyl ribofuranose; 2,4-bis-trimethylsilyl-5-methyl-uracil, a Friedel-Crafts catalyst, and hexamethyldisilizane; an eluent; an inlet for receiving [¹⁸F]-fluoride produced via a cyclotron; and an [¹⁸F]FMAU collection device.

[¹⁸F]FMAU is one of the best radiotracers for detecting cellular proliferation.

A structure of [¹⁸F]FMAU is as follows:

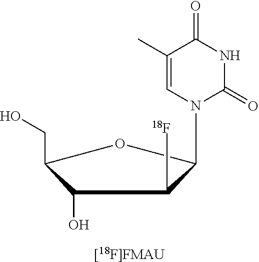

This depicted structure of [¹⁸F]FMAU is its beta-anomer, which is the preferred one in some embodiments. [¹⁸F]FMAU can be synthesized, as described herein, under CGMP-compliant conditions using the disclosed labeling kits.

As used herein, “a Friedel-Crafts catalyst” refers to any catalyst required for a Friedel-Crafts reaction. Friedel-Crafts reaction are a set of reactions developed by Charles Friedel and James Crafts in 1877 to attach substituents to an aromatic ring. Friedel-Crafts reactions are of two main types: alkylation reactions and acylation reactions. Both proceed by electrophilic aromatic substitution. Examples of Friedel-Crafts catalyst include, but are not limited to trimethylsilyl trifluoromethanesulfonate, Al Cl₃, SnCl₄, and ZnCl₂.

In one aspect, the Friedel-Crafts catalyst is trimethylsilyl trifluoromethanesulfonate (TMSOTf).

In various aspects, the system may be configured for automated one-pot synthesis.

The One-Pot Synthesis of [¹⁸F]FMAU:

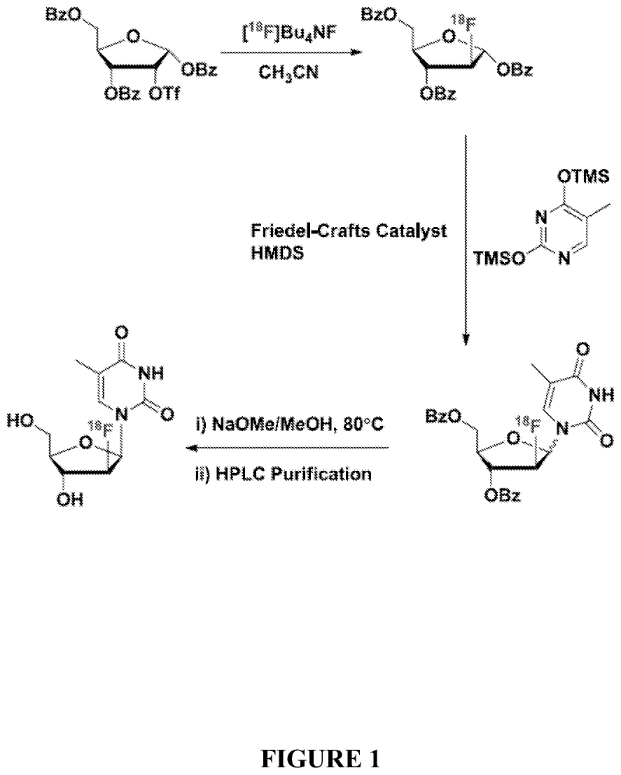

In one aspect, the present disclosure provides a one-pot reaction for [¹⁸F]FMAU synthesis. In an embodiment, the reaction starts with conversion of 2-trifluoromethane-sulfonyl-1,3,5-tri-O-benzoyl ribofuranose to 2-[¹⁸F]fluoro-1,3,5-tri-O-benzoyl arabinofuranose through the use of tetrabutylammonium fluoride and acetonitrile (e.g., at 80° C. for 20 min). The reaction then proceeds with conversion of the 2-[¹⁸F]fluoro-1,3,5-tri-O-benzoyl arabinofuranose to 2′-deoxy-2′-[¹⁸F]fluoro-3′,5′-di-O-benzoyl-5-methyl-1-beta-D-arabinofuranosyl-uracil through the use of 2,4-bis-trimethylsilyl-5-methyl-uracil, a Friedel-Crafts catalyst, and hexamethyldisilizane. Thus obtained 2′-deoxy-2′-[¹⁸F]fluoro-3′,5′-di-O-benzoyl-5-methyl-1-beta-D-arabinofuranosyl-uracil is then converted to [¹⁸F]FMAU through the use of sodium methoxide and methanol. A final HPLC purification then yields the desired pure [¹⁸F]FMAU. A scheme depicting the reaction is provided in FIG. 1.

This reaction solves several problems. For example, due to having few steps, it can be more easily controlled than the previously available methods. Concomitantly with that, it suffers from fewer production failures. In addition, it is compatible with the labeling kits disclosed herein, and can be employed within an automated synthesis module.

Various compounds may be substituted for the ones disclosed. For example, as a Friedel-Crafts catalyst, instead of trimethylsilyl trifluoromethanesulfonate, one may also use Al Cl₃, SnCl₄, or ZnCl₂. Similarly, many alternatives will be apparent to one of skill in the art to the radiofluorination reagents tetrabutylammonium fluoride and acetonitrile, as well as to the protecting group hydrolyzation reagents sodium methoxide and methanol.

In many aspects, the system is in compliance with Current Good Manufacturing Practices (CGPMs).

The system can be configured for automated one-pot synthesis, alternatively or simultaneously, the system can be in compliance with Current Good Manufacturing Practices (CGPMs).

In some aspects, the system further includes tetrabutylammonium fluoride and acetonitrile. In other aspects, the system further includes sodium methoxide and methanol.

In addition, in some aspects, the system further includes a carrier, excipient, diluent, or a combination thereof.

By “pharmaceutically acceptable” it is meant that the carrier, diluent or excipient must be compatible with the other ingredients of the formulation and not deleterious to the recipient thereof. Pharmaceutically acceptable carriers, excipients or stabilizers are well known in the art, for example from Remington's Pharmaceutical Sciences, 16th edition, Osol, A. Ed. (1980). Pharmaceutically acceptable carriers, excipients, or stabilizers are nontoxic to recipients at the dosages and concentrations employed, and may include buffers such as phosphate, citrate, and other organic acids; antioxidants including ascorbic acid and methionine; preservatives such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol; low molecular weight (less than about 10 residues) polypeptides; proteins such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes such as Zn-protein complexes; non-ionic surfactants such as TWEEN™, PLURONICS™, or polyethylene glycol (PEG); or combinations thereof.

The compounds of the present invention can exist as therapeutically acceptable salts. The present invention includes compounds listed above in the form of salts, including acid addition salts. Suitable salts include those formed with both organic and inorganic acids. Such acid addition salts will normally be pharmaceutically acceptable. However, salts of non-pharmaceutically acceptable salts may be of utility in the preparation and purification of the compound in question. Basic addition salts may also be formed and be pharmaceutically acceptable. For a more complete discussion of the preparation and selection of salts, refer to Pharmaceutical Salts: Properties, Selection, and Use (Stahl, P. Heinrich. Wiley-VCHA, Zurich, Switzerland, 2002), the entire contents of which are herein incorporated by reference.

In another embodiment, the invention provides an automated synthesis module (ASM) for synthesizing [¹⁸F]FMAU including a first container for holding 2-trifluoromethane-sulfonyl-1,3,5-tri-O-benzoyl ribofuranose; a second container for holding 2,4-bis-trimethylsilyl-5-methyl-uracil, a Friedel-Crafts catalyst, and hexamethyldisilizane; a third container for holding an eluent; an inlet for receiving [18F]-fluoride produced via a cyclotron; and a fourth container for collecting [18F]FMAU.

In one aspect, the Friedel-Crafts catalyst is trimethylsilyl trifluoromethanesulfonate (TMSOTf).

In various aspects, the ASM is configured for automated one-pot synthesis. In many aspects, the ASM is in compliance with CGPMs. The ASM can be configured for automated one-pot synthesis, alternatively or simultaneously, the ASM can be in compliance with CGPMs.

In some aspects, the ASM further includes a fifth container for holding tetrabutylammonium fluoride and acetonitrile. In other aspects, the ASM further includes a sixth container for holding sodium methoxide and methanol. In some aspects, the ASM further includes a seventh container for holding carrier, excipient, diluent, or a combination thereof.

In an additional embodiment, the invention provides a method of synthesizing [¹⁸F]FMAU in a one-pot reaction including incubating 2-trifluoromethane-sulfonyl-1,3,5-tri-O-benzoyl ribofuranose with an [¹⁸F]-containing compound, thereby generating 2-[¹⁸F]fluoro-1,3,5-tri-O-benzoyl ribofuranose; incubating the 2-[¹⁸F]fluoro-1,3,5-tri-O-benzoyl ribofuranose with a solution containing 2,4-bis-trimethylsilyl-5-methyl-uracil, a Friedel-Crafts catalyst, and hexamethyldisilizane, thereby generating a mixture; and purifying the mixture via HPLC, thereby obtaining [¹⁸F]FMAU.

In various aspects, the method further includes, before purifying the mixture via HPLC, incubating the mixture with sodium methoxide and methanol to remove benzoyl groups. In other aspects, the method further includes adding a carrier, excipient, diluent, or a combination thereof to the [¹⁸F]FMAU. In some aspects, the method further includes diluting a solution of the [¹⁸F]FMAU to less than or equal to about 25 mCi per unit dose. In one aspect, the method is performed in a CGMP-compliant environment. In other aspects, the method is performed in an automated synthesis module. The method can be configured for automated one-pot synthesis, alternatively or simultaneously, the method can be in compliance with CGPMs. In one aspect, the Friedel-Crafts catalyst is trimethylsilyl trifluoromethanesulfonate (TMSOTf). In many aspects, the [¹⁸F]-containing compound is [¹⁸F]tetrabutylammonium fluoride.

In yet another embodiment, the invention provides a method of screening conditions for GMP-compliant one-pot synthesis of [¹⁸F]FMAU including incubating in multiple ASMs or in one ASM at different times, an amount of 2-trifluoromethane-sulfonyl-1,3,5-tri-O-benzoyl ribofuranose with an amount of [18F]-containing compound, thereby generating an amount of 2-[18F]fluoro-1,3,5-tri-O-benzoyl ribofuranose; incubating, in each of the multiple ASMs or in one ASM at each of the different times, an amount of the 2-[18F]fluoro-1,3,5-tri-O-benzoyl ribofuranose with a solution containing an amount of 2,4-bis-trimethylsilyl-5-methyl-uracil, an amount of a Friedel-Crafts catalyst, and an amount of hexamethyldisilizane using pre-selected solvents, solute concentrations, incubation times, or temperatures thereby generating an amount of a mixture; purifying, in each of the multiple ASMs or in one ASM at each of the different times, an amount of the mixture via HPLC, thereby obtaining an amount of [18F]FMAU; and determining the amount of [¹⁸F]FMAU obtained using each of the pre-selected solvents, solute concentrations, incubation times, or temperatures.

In one aspect, the Friedel-Crafts catalyst is trimethylsilyl trifluoromethanesulfonate (TMSOTf).

In many aspects, the method is carried out in a CGPM-compliant environment. In some aspects, the screening methods are performed under one-pot synthesis conditions. The methods can be configured for automated one-pot synthesis, alternatively or simultaneously, the methods can be in compliance with CGPMs.

In some aspects, the screening methods further include, before purifying the mixture via HPLC, incubating the mixture with sodium methoxide and methanol to remove benzoyl groups.

In one embodiment, the invention provides a method of constructing a labeling system for obtaining [¹⁸F]FMAU including providing a first container of the system containing 2-trifluoromethane-sulfonyl-1,3,5-tri-O-benzoyl ribofuranose; providing a second container of the system containing 2,4-bis-trimethylsilyl-5-methyl-uracil, a Friedel-Crafts catalyst, and hexamethyldisilizane, wherein the second container is disposed to communicate with the first container; providing a third container of the system containing an eluent, wherein the third container is disposed to communicate with the first container; connecting to the system an inlet for receiving [18F]-fluoride produced via a cyclotron, wherein the inlet is disposed to communicate with the third container; and providing a fourth container of the system for collecting [18F]FMAU, wherein the fourth container is disposed to communicate with the second container.

Labeling Kits for Synthesis of [¹⁸F]FMAU:

Also provided as aspects of the present invention are labeling kits for [¹⁸F]FMAU synthesis. The labeling kits can be assembled in an automated synthesis system, after which various reaction conditions can be investigated or optimized.

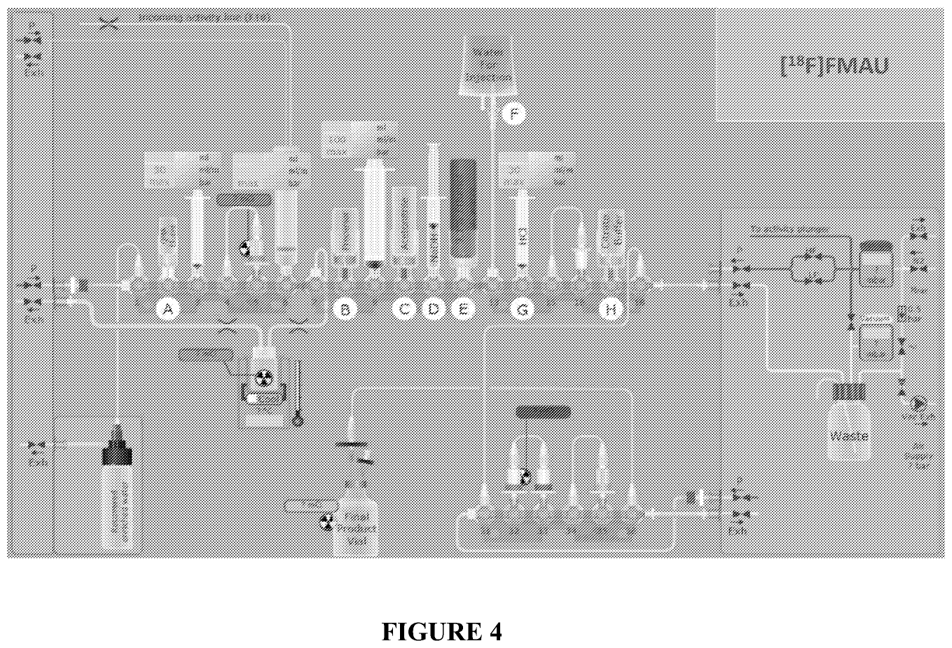

FIG. 4 shows an embodiment of the labeling kit for automated manufacture of [¹⁸F]FMAU in full compliance with cGMP environment. Shown in the figure are arranged containers for holding the QMA eluent, the precursor (e.g., the sugar precursor), acetonitrile, sodium hydroxide, ethanol in water, hydrochloric acid, citrate buffer, water for injection, recovered enriched water, waste, and the final product. An incoming activity line brings in ¹⁸F generated at a cyclotron. In some embodiments, display units can show various measurements of the pressure, flow rate, as well as volume.

In one aspect, the Friedel-Crafts catalyst is trimethylsilyl trifluoromethanesulfonate (TMSOTf).

In many aspects, the system is configured for automated one-pot synthesis. In other aspects, the system is in compliance with CGPMs. The method can be configured for automated one-pot synthesis, alternatively or simultaneously, the method can be in compliance with CGPMs.

In one aspect, the method further includes providing a fifth container of the system containing tetrabutylammonium fluoride and acetonitrile, wherein the fifth container is disposed to communicate with the first container. In another aspect, the method further includes providing a sixth container of the system containing sodium methoxide and methanol, wherein the sixth container is disposed to communicate with the third container. In some aspects, the method further includes providing a seventh container of the system containing a carrier, excipient, diluent, or a combination thereof, wherein the seventh container is disposed to communicate with the fourth container.

In another embodiment, the invention provides a method of detecting cellular proliferation via PET imaging including incubating 2-trifluoromethane-sulfonyl-1,3,5-tri-O-benzoyl ribofuranose with an [18F]-containing compound, thereby generating 2-[18F]fluoro-1,3,5-tri-O-benzoyl ribofuranose; incubating the 2-[18F]fluoro-1,3,5-tri-O-benzoyl ribofuranose with a solution containing 2,4-bis-trimethylsilyl-5-methyl-uracil, a Friedel-Crafts catalyst, and hexamethyldisilizane, thereby generating [¹⁸F]FMAU; administering the [¹⁸F]FMAU to a subject; and detecting the [¹⁸F]FMAU by imaging an area of the subject via PET. In some aspects, the [¹⁸F]FMAU is administered to the subject at less than or equal to 25 mCi per unit dose.

The term “cancer” refers to a group diseases characterized by abnormal and uncontrolled cell proliferation starting at one site (primary site) with the potential to invade and to spread to other sites (secondary sites, metastases) which differentiate cancer (malignant tumor) from benign tumor. Virtually all the organs can be affected, leading to more than 100 types of cancer that can affect humans. Cancers can result from many causes including genetic predisposition, viral infection, exposure to ionizing radiation, exposure environmental pollutant, tobacco and or alcohol use, obesity, poor diet, lack of physical activity or any combination thereof. “Metastasis” refers to the biologically process involved in the development of metastases. “Neoplasm” or “tumor” including grammatical variations thereof means new and abnormal growth of tissue, which may be benign or cancerous.

Exemplary cancers include breast cancer, non-small cell lung cancer, brain cancer, and osteosarcoma. Exemplary cancers also include, but are not limited to, Acute Lymphoblastic Leukemia, Adult; Acute Lymphoblastic Leukemia, Childhood; Acute Myeloid Leukemia, Adult; Adrenocortical Carcinoma; Adrenocortical Carcinoma, Childhood; AIDS-Related Lymphoma; AIDS-Related Malignancies; Anal Cancer; Astrocytoma, Childhood Cerebellar; Astrocytoma, Childhood Cerebral; Bile Duct Cancer, Extrahepatic; Bladder Cancer; Bladder Cancer, Childhood; Bone Cancer, Osteosarcoma/Malignant Fibrous Histiocytoma; Brain Stem Glioma, Childhood; Brain Tumor, Adult; Brain Tumor, Brain Stem Glioma, Childhood; Brain Tumor, Cerebellar Astrocytoma, Childhood; Brain Tumor, Cerebral Astrocytoma/Malignant Glioma, Childhood; Brain Tumor, Ependymoma, Childhood; Brain Tumor, Medulloblastoma, Childhood; Brain Tumor, Supratentorial Primitive Neuroectodermal Tumors, Childhood; Brain Tumor, Visual Pathway and Hypothalamic Glioma, Childhood; Brain Tumor, Childhood (Other); Breast Cancer; Breast Cancer and Pregnancy; Breast Cancer, Childhood; Breast Cancer, Male; Bronchial Adenomas/Carcinoids, Childhood: Carcinoid Tumor, Childhood; Carcinoid Tumor, Gastrointestinal; Carcinoma, Adrenocortical; Carcinoma, Islet Cell; Carcinoma of Unknown Primary; Central Nervous System Lymphoma, Primary; Cerebellar Astrocytoma, Childhood; Cerebral Astrocytoma/Malignant Glioma, Childhood; Cervical Cancer; Childhood Cancers; Chronic Lymphocytic Leukemia; Chronic Myelogenous Leukemia; Chronic Myeloproliferative Disorders; Clear Cell Sarcoma of Tendon Sheaths; Colon Cancer; Colorectal Cancer, Childhood; Cutaneous T-Cell Lymphoma; Endometrial Cancer; Ependymoma, Childhood; Epithelial Cancer, Ovarian; Esophageal Cancer; Esophageal Cancer, Childhood; Ewing's Family of Tumors; Extracranial Germ Cell Tumor, Childhood; Extragonadal Germ Cell Tumor; Extrahepatic Bile Duct Cancer; Eye Cancer, Intraocular Melanoma; Eye Cancer, Retinoblastoma; Gallbladder Cancer; Gastric (Stomach) Cancer; Gastric (Stomach) Cancer, Childhood; Gastrointestinal Carcinoid Tumor; Germ Cell Tumor, Extracranial, Childhood; Germ Cell Tumor, Extragonadal; Germ Cell Tumor, Ovarian; Gestational Trophoblastic Tumor; Glioma. Childhood Brain Stem; Glioma. Childhood Visual Pathway and Hypothalamic; Hairy Cell Leukemia; Head and Neck Cancer; Hepatocellular (Liver) Cancer, Adult (Primary); Hepatocellular (Liver) Cancer, Childhood (Primary); Hodgkin's Lymphoma, Adult; Hodgkin's Lymphoma, Childhood; Hodgkin's Lymphoma During Pregnancy; Hypopharyngeal Cancer; Hypothalamic and Visual Pathway Glioma, Childhood; Intraocular Melanoma; Islet Cell Carcinoma (Endocrine Pancreas); Kaposi's Sarcoma; Kidney Cancer; Laryngeal Cancer; Laryngeal Cancer, Childhood; Leukemia, Acute Lymphoblastic, Adult; Leukemia, Acute Lymphoblastic, Childhood; Leukemia, Acute Myeloid, Adult; Leukemia, Acute Myeloid, Childhood; Leukemia, Chronic Lymphocytic; Leukemia, Chronic Myelogenous; Leukemia, Hairy Cell; Lip and Oral Cavity Cancer; Liver Cancer, Adult (Primary); Liver Cancer, Childhood (Primary); Lung Cancer, Non-Small Cell; Lung Cancer, Small Cell; Lymphoblastic Leukemia, Adult Acute; Lymphoblastic Leukemia, Childhood Acute; Lymphocytic Leukemia, Chronic; Lymphoma, AIDS—Related; Lymphoma, Central Nervous System (Primary); Lymphoma, Cutaneous T-Cell; Lymphoma, Hodgkin's, Adult; Lymphoma, Hodgkin's; Childhood; Lymphoma, Hodgkin's During Pregnancy; Lymphoma, Non-Hodgkin's, Adult; Lymphoma, Non-Hodgkin's, Childhood; Lymphoma, Non-Hodgkin's During Pregnancy; Lymphoma, Primary Central Nervous System; Macroglobulinemia, Waldenstrom's; Male Breast Cancer; Malignant Mesothelioma, Adult; Malignant Mesothelioma, Childhood; Malignant Thymoma; Medulloblastoma, Childhood; Melanoma; Melanoma, Intraocular; Merkel Cell Carcinoma; Mesothelioma, Malignant; Metastatic Squamous Neck Cancer with Occult Primary; Multiple Endocrine Neoplasia Syndrome, Childhood; Multiple Myeloma/Plasma Cell Neoplasm; Mycosis Fungoides; Myelodysplasia Syndromes; Myelogenous Leukemia, Chronic; Myeloid Leukemia, Childhood Acute; Myeloma, Multiple; Myeloproliferative Disorders, Chronic; Nasal Cavity and Paranasal Sinus Cancer; Nasopharyngeal Cancer; Nasopharyngeal Cancer, Childhood; Neuroblastoma; Non-Hodgkin's Lymphoma, Adult; Non-Hodgkin's Lymphoma, Childhood; Non-Hodgkin's Lymphoma During Pregnancy; Non-Small Cell Lung Cancer; Oral Cancer, Childhood; Oral Cavity and Lip Cancer; Oropharyngeal Cancer; Osteosarcoma/Malignant Fibrous Histiocytoma of Bone; Ovarian Cancer, Childhood; Ovarian Epithelial Cancer; Ovarian Germ Cell Tumor; Ovarian Low Malignant Potential Tumor; Pancreatic Cancer; Pancreatic Cancer, Childhood', Pancreatic Cancer, Islet Cell; Paranasal Sinus and Nasal Cavity Cancer; Parathyroid Cancer; Penile Cancer; Pheochromocytoma; Pineal and Supratentorial Primitive Neuroectodermal Tumors, Childhood; Pituitary Tumor; Plasma Cell Neoplasm/Multiple Myeloma; Pleuropulmonary Blastoma; Pregnancy and Breast Cancer; Pregnancy and Hodgkin's Lymphoma; Pregnancy and Non-Hodgkin's Lymphoma; Primary Central Nervous System Lymphoma; Primary Liver Cancer, Adult; Primary Liver Cancer, Childhood; Prostate Cancer; Rectal Cancer; Renal Cell (Kidney) Cancer; Renal Cell Cancer, Childhood; Renal Pelvis and Ureter, Transitional Cell Cancer; Retinoblastoma; Rhabdomyosarcoma, Childhood; Salivary Gland Cancer; Salivary Gland'Cancer, Childhood; Sarcoma, Ewing's Family of Tumors; Sarcoma, Kaposi's; Sarcoma (OsteosarcomaVMalignant Fibrous Histiocytoma of Bone; Sarcoma, Rhabdomyosarcoma, Childhood; Sarcoma, Soft Tissue, Adult; Sarcoma, Soft Tissue, Childhood; Sezary Syndrome; Skin Cancer; Skin Cancer, Childhood; Skin Cancer (Melanoma); Skin Carcinoma, Merkel Cell; Small Cell Lung Cancer; Small Intestine Cancer; Soft Tissue Sarcoma, Adult; Soft Tissue Sarcoma, Childhood; Squamous Neck Cancer with Occult Primary, Metastatic; Stomach (Gastric) Cancer; Stomach (Gastric) Cancer, Childhood; Supratentorial Primitive Neuroectodermal Tumors, Childhood; T-Cell Lymphoma, Cutaneous; Testicular Cancer; Thymoma, Childhood; Thymoma, Malignant; Thyroid Cancer; Thyroid Cancer, Childhood; Transitional Cell Cancer of the Renal Pelvis and Ureter; Trophoblastic Tumor, Gestational; Unknown Primary Site, Cancer of, Childhood; Unusual Cancers of Childhood; Ureter and Renal Pelvis, Transitional Cell Cancer; Urethral Cancer; Uterine Sarcoma; Vaginal Cancer; Visual Pathway and Hypothalamic Glioma, Childhood; Vulvar Cancer; Waldenstrom's Macro globulinemia; and Wilms' Tumor.

“Cancer cell” or “tumor cell”, and grammatical equivalents refer to the total population of cells derived from a tumor or a pre-cancerous lesion, including both non tumorigenic cells, which comprise the bulk of the tumor population, and tumorigenic stem cells (cancer stem cells).

As used herein, “PET” or “PET-scan” refers to positron emission tomography (PET) scanning using a molecular tracer. PET-scan is a nuclear medicine functional imaging technique that is widely used in the medical field to observe metabolic processes in the body as an aid to the diagnosis of disease.

The terms “administration of” and “administering a” compound should be understood to mean providing a compound of the disclosure or pharmaceutical composition to a subject. An exemplary administration route is intravenous administration. In general, administration routes include but are not limited to intracutaneous, subcutaneous, intravenous, intraperitoneal, intraarterial, intrathecal, intracapsular, intraorbital, intracardiac, intradermal, transdermal, transtracheal, sub cuticular, intraarticulare, subcapsular, subarachnoid, intraspinal and intrasternal, oral, sublingual buccal, rectal, vaginal, nasal ocular administrations, as well infusion, inhalation, and nebulization. The phrases “parenteral administration” and “administered parenterally” as used herein means modes of administration other than enteral and topical administration. The compositions of the present invention may be processed in a number of ways depending on the anticipated application and appropriate delivery or administration of the pharmaceutical composition. For example, the compositions may be formulated for injection.

The compounds can be administered in various modes, e.g. orally, topically, or by injection. In some embodiments, the compounds (e.g., [¹⁸F]FMAU) are administrated by injection. The precise amount of compound administered to a patient can be determined by a person of skill in the art. The specific dose level for any particular patient will depend upon a variety of factors including the activity of the specific compound employed, the age, body weight, general health, sex, diets, time of administration, and route of administration.

The term “subject” as used herein refers to any individual or patient to which the subject methods are performed. Generally the subject is human, although as will be appreciated by those in the art, the subject may be an animal. Thus other animals, including mammals such as rodents (including mice, rats, hamsters and guinea pigs), cats, dogs, rabbits, farm animals including cows, horses, goats, sheep, pigs, etc., and primates (including monkeys, chimpanzees, orangutans and gorillas) are included within the definition of subject.

In one aspect, the Friedel-Crafts catalyst is trimethylsilyl trifluoromethanesulfonate (TMSOTf).

In some aspects, the method further includes diluting a solution of the [¹⁸F]FMAU to less than or equal to about 25 mCi per unit dose.

Presented below are examples discussing synthesis and methods of use of [¹⁸F]FMAU; contemplated for the discussed applications. The following examples are provided to further illustrate the embodiments of the present invention, but are not intended to limit the scope of the invention. While they are typical of those that might be used, other procedures, methodologies, or techniques known to those skilled in the art may alternatively be used.

### EXAMPLES

### Example 1

### [18F]FMAU Synthesis Reaction

The following describes the details of an [¹⁸F]FMAU synthesis reaction.

All reagents and solvents were purchased from Aldrich Chemical (Milwaukee, WI, USA), and used without further purification. Solid-phase extraction cartridges were purchased from Waters. Ion exchange cartridges were purchased from ABX (Germany). 2-Trifluoromethanesulfonyl-1,3,5-tri-O-benzoyl-α-D-ribofuranose (precursor) and bis-2,4-trimethylsilyl-5-methyluracil were purchased from ABX (Germany). Non-radioactive FMAU anomers were prepared in house and used as HPLC standards. Analysis was performed on an analytical reversed-phase HPLC system equipped with a dual UV absorbance detector (Waters 2487) using a phenomenex C18 RP (250×4.6 mm 5 micron). [¹⁸F]FMAU purification was performed on an isocratic HPLC with UV detector operated at 254 nm and radioactivity detector. A semipreparative C18 reverse phase column (phenomenex C18, 250×10 mm, 10 μm) was used in the separation. A solution of 6% ethanol in phosphate buffer (10 mM, pH 6.5) or 8% MeCN/water was used for the purification of [18 F]-FMAU. A solution of 8% MeCN in water was used for the quality control of [18 F]-FMAU on an analytical HPLC.

The solutions of potassium carbonate and Kryptofix K2.2.2[or tetrabutylammonium bicarbonate (TBAB) and MeCN] were loaded into Reservoirs, respectively. Other Reservoirs were filled with precursor 1 (5.0-10 mg sugar triflate in 600 μl anhydrous MeCN), precursor 2 [a solution of 20 mg TMS-uracil, 100 μl hexamethyldisilizane (HMDS), and 150 μl trimethylsilyl trifluoromethanesulfonate (TMSOTf), in 300 μl dichloroethane], KOMe solution (0.4 ml, 2.0 N in MeOH), and HCl (0.2 ml, 4.0 N HCl+1.0 ml HPLC solvent), respectively. The target water containingl 18 F was passed through a preconditioned QMA cartridge where the 18 F-F-was trapped. The 18 F was released from the QMA cartridge by passing K2CO3 or TBAB solution through the cartridge and allowed to enter into the reactor. Kryptofix solution or MeCN was added into the reactor and the whole mixture was dried at 95° C. in combination of nitrogen flow and vacuum. The precursor solution was added to the dried 18 F ion and heated at 80° C. for 20 min. The MeCN was then evaporated and precursor 2 solution was added to the reactor. The reaction mixture was heated for 1 h at 85° C. The solvent was removed and KOMe solution was then added. The mixture was heated for 7 min at 80° C. and MeOH was removed under vacuum. The HCl and mobile phase solution was then added to the reactor and passed through an alumina cartridge to a V-vial. The crude product solution was loaded on HPLC and the column was eluted with 6% EtOH/phosphate buffer (10 mM,pH6.5) or 8% MeCN/water at 4 ml/min. The appropriate fraction containing [¹⁸F]FMAU(17.4min) was collected into the collection flask, which was then transferred to the receiving vial after filtered through a Millipore filter. Rotary evaporation was performed first if MeCN/water was used as the eluent. The radioactivity of the final product was then measured. On analytical HPLC, [¹⁸F]FMAU has a retention time of 9.3 min when 8% MeCN in water was used as the mobile phase.

### Example 2

### Micropet/Ct Imaging of a Mouse Using [¹⁸F]FMAU

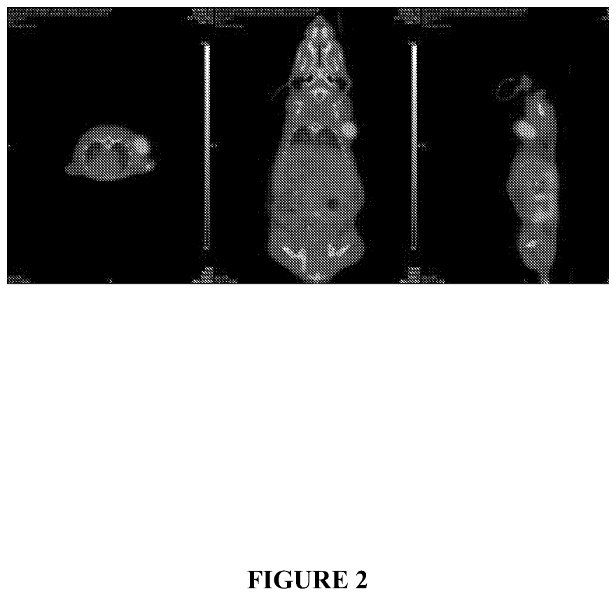

We studied the [¹⁸F]FMAU obtained through our synthesis method using the labeling kit in animal models. Some of our results are shown in FIG. 2. FIG. 2 shows three panels of images obtained from microPET/Ct imaging of a mouse bearing MDA-MB-231 breast tumor at 1 hour post-injection of [¹⁸F]FMAU.

### Example 3

### Breast and Prostate Cancer Patients Imaging Using [¹⁸F]FMAU

In addition to animal models, we also studied the [¹⁸F]FMAU obtained through our synthesis method using the labeling kit in patients (Phase I) with known breast and prostate cancer.

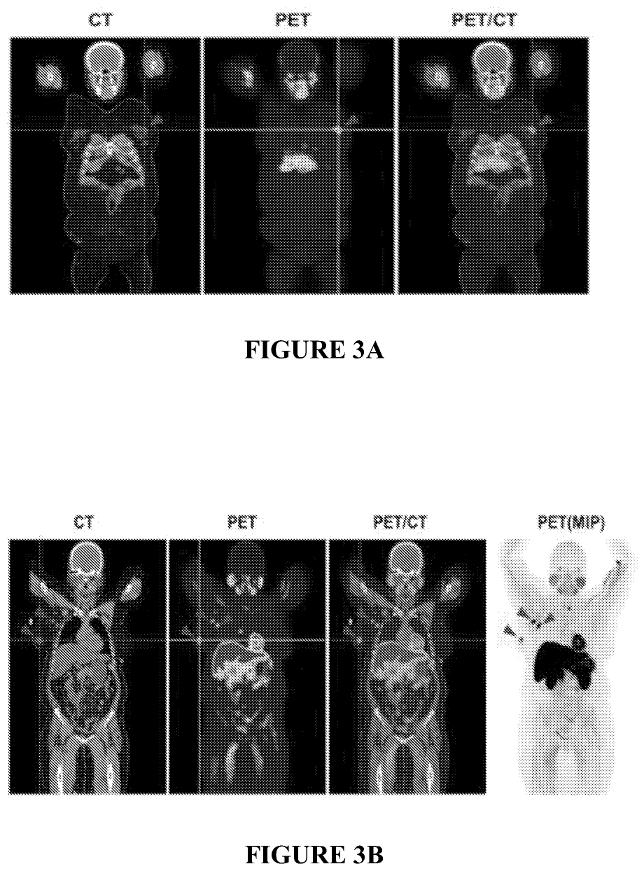

For example, in breast cancer patients, the PET imaging showed excellent primary breast tumor as well as metastatic disease uptake of [¹⁸F]FMAU. This is shown in FIG. 3. No adverse reactions were observed for all studied patients. No major circulating metabolites were identified in human blood at 1 hour post-injection of [¹⁸F]FMAU. In view of the promising clinical Phase I data of [¹⁸F]FMAU, it is clear that FMAU is a promising candidate for PET imaging of tumor cell proliferation, and may ultimately complement the role of FLT or other cell proliferation markers currently under development.

Although the invention has been described with reference to the above examples, it will be understood that modifications and variations are encompassed within the spirit and scope of the invention. Accordingly, the invention is limited only by the following claims.

1. A system for producing 2′-deoxy-2′-[¹⁸F]fluoro-5-methyl-1-beta-D-arabinofuranosyl-uracil ([¹⁸F]FMAU) comprising:
2-trifluoromethane-sulfonyl-1,3,5-tri-O-benzoyl ribofuranose;
2,4-bis-trimethylsilyl-5-methyl-uracil, a Friedel-Crafts catalyst, and hexamethyldisilizane;
an eluent;
an inlet for receiving [¹⁸F]-fluoride fluoride produced via a cyclotron; and
an [¹⁸F]FMAU collection device.

2. The system of claim 1, wherein the Friedel-Crafts catalyst is trimethylsilyl trifluoromethanesulfonate.

3. The system of claim 1, wherein the system is configured for automated one-pot synthesis.

4. The system of claim 1, wherein the system is in compliance with CGPMs.

5. The system of claim 1, further comprising tetrabutylammonium fluoride and acetonitrile.

6. The system of claim 1, further comprising sodium methoxide and methanol.

7. The system of claim 1, further comprising a carrier, excipient, diluent, or a combination thereof.

8. An automated synthesis module (ASM) for synthesizing 2′-deoxy-2′[¹⁸F]fluoro-5-methyl-1-beta-D-arabinofuranosyl-uracil ([¹⁸F]FMAU) in compliance with CGMPs comprising:
a first container for holding 2-trifluoromethane-sulfonyl-1,3,5-tri-O-benzoyl ribofuranose;
a second container for holding 2,4-bis-trimethylsilyl-5-methyl-uracil, a Friedel-Crafts catalyst, and hexamethyldisilizane;
a third container for holding an eluent;
an inlet for receiving [¹⁸F]-fluoride produced via a cyclotron; and
a fourth container for collecting [¹⁸F]FMAU.

9. The ASM of claim 8, wherein the Friedel-Crafts catalyst is trimethylsilyl trifluoromethanesulfonate.

10. The ASM of claim 8, wherein the system is configured for automated one-pot synthesis.

11. The ASM of claim 8, wherein the ASM is in compliance with CGPMs.

12. The ASM of claim 8, further comprising a fifth container for holding tetrabutylammonium fluoride and acetonitrile.

13. The ASM of claim 8, further comprising a sixth container for holding sodium methoxide and methanol.

14. The ASM of claim 8, further comprising a seventh container for holding carrier, excipient, diluent, or a combination thereof.

15. A method of synthesizing 2′-deoxy-2′[¹⁸F]fluoro-5-methyl-1-beta-D-arabinofuranosyl-uracil ([¹⁸F]FMAU) in a one-pot reaction comprising:
incubating 2-trifluoromethane-sulfonyl-1,3,5-tri-O-benzoyl ribofuranose with an [¹⁸F]-containing compound, thereby generating 2-[¹⁸F]fluoro-1,3,5-tri-O-benzoyl ribofuranose;
incubating the 2-[¹⁸F]fluoro-1,3,5-tri-O-benzoyl ribofuranose with a solution containing 2,4-bis-trimethylsilyl-5-methyl-uracil, a Friedel-Crafts catalyst, and hexamethyldisilizane, thereby generating a mixture; and
purifying the mixture via HPLC, thereby obtaining [¹⁸F]FMAU.

16. The method of claim 15, further comprising, before purifying the mixture via HPLC, incubating the mixture with sodium methoxide and methanol to remove benzoyl groups.

17. The method of claim 15, further comprising adding a carrier, excipient, diluent, or a combination thereof to the [¹⁸F]FMAU.

18. The method of claim 15, further comprising diluting a solution of the [¹⁸F]FMAU to less than or equal to about 25 mCi per unit dose.

19. The method of claim 15, wherein the method is carried out in a CGMP-compliant environment.

20. The method of claim 15, wherein the method is performed in an automated synthesis module.

21. The method of claim 15, wherein the Friedel-Crafts catalyst is trimethylsilyl trifluoromethanesulfonate.

22. The method of claim 15, wherein the [¹⁸F]-containing compound is [¹⁸F]tetrabutylammonium fluoride.

23-34. (canceled)

35. A method of detecting cellular proliferation via PET imaging comprising:
incubating 2-trifluoromethane-sulfonyl-1,3,5-tri-O-benzoyl ribofuranose with an [¹⁸F]-containing compound, thereby generating 2-[¹⁸F]fluoro-1,3,5-tri-O-benzoyl ribofuranose;
incubating the 2-[¹⁸F]fluoro-1,3,5-tri-O-benzoyl ribofuranose with a solution containing 2,4-bis-trimethylsilyl-5-methyl-uracil, a Friedel-Crafts catalyst, and hexamethyldisilizane, thereby generating a [¹⁸F]FMAU;
administering the [¹⁸F]FMAU to a subject; and
detecting the [¹⁸F]FMAU by imaging an area of the subject via PET.

36. The method of claim 35, wherein the Friedel-Crafts catalyst is trimethylsilyl trifluoromethanesulfonate.

37. The method of claim 35, wherein the [¹⁸F]FMAU is administered to the subject at less than or equal to 25 mCi per unit dose.
